(19) United States
(12) Patent Application Publication
(10) Pub. No.: US 2022/0219984 A1
(43) Pub. Date: Jul. 14, 2022

(54) PROCESS FOR PREPARING AMMONIUM SALT CONTAINING A FLUOROSULFONYL GROUP

(71) Applicant: ARKEMA FRANCE, Colombes (FR)

(72) Inventors: Grégory SCHMIDT, PIERRE-BENITE Cedex (FR); Philippe LEDUC, PIERRE-BENITE Cedex (FR); Rémy TEISSIER, PIERRE-BENITE Cedex (FR)

(21) Appl. No.: 17/611,950

(22) PCT Filed: May 19, 2020
(86) PCT No.: PCT/FR2020/050828

§ 371 Date: Nov. 17, 2021

(30) Foreign Application Priority Data
May 22, 2019 (FR) FR1905385

(51) Int. Cl. C01B 21/086

(52) U.S. Cl. CPC C01B 21/086

(57) ABSTRACT

The present invention relates to a process for preparing a compound having the following formula (II):

F—SO₂—N⁻—SO₂—R₁NH₄⁺ (II)

wherein R₁ represents F or a linear or branched alkyl radical, substituted with at least one fluorine atom, said process comprising a step of bringing an anhydrous flow F1 comprising ammonia (NH₃) into contact with a compound of formula (I):

F—SO₂—NH—SO₂—R₁ (I)

R₁ is as defined above.

### FIELD OF THE INVENTION

The present invention relates to a process for preparing ammonium salts containing a fluorosulfonyl group.

The present invention also relates to a process for preparing lithium salts of imides containing a fluorosulfonyl group.

### TECHNICAL BACKGROUND

By virtue of their very low basicity, anions of sulfonylimide type are increasingly used in the field of energy storage in the form of inorganic salts in batteries, or of organic salts in supercapacitors or in the field of ionic liquids. Since the battery market is booming and the reduction of battery manufacturing costs is becoming a major issue, a large-scale, low-cost synthesis process for anions of this type is required.

In the specific field of Li-ion batteries, the salt currently most widely used is LiPF₆, but this salt exhibits many disadvantages, such as limited thermal stability, sensitivity to hydrolysis, and therefore lower battery safety. Recently, new salts bearing the FSO₂⁻ group have been studied and have demonstrated many advantages, such as better ion conductivity and resistance to hydrolysis. One of these salts, LiFSI (LiN(FSO₂)₂), has shown highly advantageous properties that make it a good candidate for replacing LiPF₆.

There are various processes for preparing LiFSI. The examples in EP2505551 describe in particular the fluorination of a bis(chlorosulfonyl)imide with a fluorinating agent ZnF₂ to form a zinc bis(fluorosulfonyl)imide salt. The zinc salt is then contacted with an aqueous ammonia solution to form an ammonium bis(fluorosulfonyl)imide salt. A cation exchange step is carried out with LiOH to obtain LiFSI.

This process has the disadvantage of using an aqueous solution, which has the effect of solubilizing the LiFSI. In order to recover the dissolved LiFSI, the process includes additional extraction steps, which complicates the process and impacts production costs.

Moreover, this process for preparing LiFSI includes the preparation of several intermediate compounds (zinc salt, ammonium salt). The accumulation of steps can give rise to a reduction in the final yields of LiFSI.

There is therefore still a need for a process for preparing the lithium salt of bis(fluorosulfonyl)imide that does not have at least one of the abovementioned disadvantages.

### DESCRIPTION OF THE INVENTION

The present invention relates to a process for preparing a compound of the following formula (II):

F—SO₂—N⁻—SO₂—R₁NH₄⁺ (II)

wherein R₁ represents F or a linear or branched alkyl radical substituted by at least one fluorine atom, said process comprising a step of contacting an anhydrous stream F1 comprising ammonia (NH₃) with a compound of formula (I):

F—SO₂—NH—SO₂—R₁ (I)

R¹ being as defined above.

In the context of the invention and unless otherwise stated, “anhydrous stream” is understood as meaning a stream having a water content of less than 800 ppm, preferably less than or equal to 500 ppm, and advantageously less than or equal to 200 ppm.

In one embodiment, R¹ represents one of the following radicals: F, CF₃, CHF₂, CH₂F, C₂HF₄, C₂H₂F₃, C₂H₃F₂, C₂F₉, C₃F₇, C₃H₂F₅, C₃H₄F₃, C₄F₉, C₄H₂F₇, C₄H₄F₉, or C₅F₁₁, R¹ preferably being F.

The anhydrous stream F1 may be a liquid anhydrous stream or a gaseous anhydrous stream.

When the anhydrous stream F1 is a liquid stream, it may be a stream comprising liquid ammonia (NH₃) or comprising a solution of ammonia (NH₃) in an organic solvent or mixture of organic solvents.

The organic solvent may be chosen from the group consisting of esters, nitriles, ethers, amines, phosphines, and mixtures thereof.

The organic solvent is preferably chosen from the group consisting of methyl acetate, ethyl acetate, butyl acetate, acetonitrile, propionitrile, isobutyronitrile, glutaronitrile, dioxane, tetrahydrofuran, methanol, ethanol, propanol, butanol, and mixtures thereof.

Preferably, the organic solvent is butyl acetate.

The process may comprise, prior to the abovementioned contacting step, a step of dissolving gaseous or liquid NH₃ in an organic solvent or mixture of organic solvents as defined above, advantageously forming an anhydrous liquid stream F1.

The concentration of ammonia (NH₃) dissolved in an organic solvent or mixture of organic solvents may be between 0.01 mol/L and the maximum solubility of ammonia in said organic solvent(s).

When the anhydrous stream F1 is a gaseous stream, it comprises gaseous ammonia (NH₃).

The abovementioned contacting step may be performed at a temperature T ranging from 0° C. to 40° C., preferably from 0° C. to 30° C., and preferentially from 2° C. to 30° C.

The abovementioned contacting step may be performed at a pressure P of between 0.1 and 15 bar absolute.

The molar ratio of compound of formula (I) to ammonia (NH₃) may be between 0.01 and 1, preferably between 0.1 and 0.5, and advantageously between 0.1 and 0.4.

The abovementioned compound of formula (I) may be obtained by a process comprising a step of fluorination of a compound of formula (A):

Cl—(SO₂)—NH—(SO₂)—R² (A)

where R² represents one of the following radicals: Cl, F, CF₃, CHF₂, CH₂F, C₂HF₄, C₂H₂F₃, C₂H₃F₂, C₂F₅, C₃F₇, C₃H₄F₃, C₃HF₆, C₄F₃, C₄H₂F₇, C₄H₄F₅, C₅F₁₁, C₆F₁₃, C₇F₁₅, C₅F₁₇ or C₃F₁₉, R₂ preferably representing Cl;
with at least one fluorinating agent.

The fluorinating agent may be chosen from the group consisting of HF (for example anhydrous HF), KF, AsF₃, BiF₃, ZnF₂, SnF₂, PbF₂, CuF₂, and mixtures thereof, the fluorinating agent preferably being HF and even more preferentially anhydrous HF.

In the context of the invention, the term “anhydrous HF” is understood as meaning HF containing less than 500 ppm of water, preferably less than 300 ppm of water, more preferably less than 200 ppm of water.

This step may be carried out in at least one organic solvent OS1. The organic solvent OS1 preferably has a donor number of between 1 and 70 and advantageously of between 5 and 65. The donor number of a solvent represents the value −ΔH, ΔH being the enthalpy of the interaction between the solvent and antimony pentachloride (according to the method described in Journal of Solution Chemistry, vol. 13, No. 9, 1984). Organic solvent OS1 may in particular be esters, nitriles, dinitriles, ethers, diethers, amines, phosphines, and mixtures thereof.

The organic solvent OS1 is preferably chosen from the group consisting of methyl acetate, ethyl acetate, butyl acetate, acetonitrile, propionitrile, isobutyronitrile, glutaronitrile, dioxane, tetrahydrofuran, triethylamine, tripropylamine, diethylisopropylamine, pyridine, trimethylphosphine, triethylphosphine, diethylisopropylphosphine, and mixtures thereof. In particular, the organic solvent OS1 is dioxane or butyl acetate.

The fluorination step may be performed at a temperature between 0° C. and the boiling point of the organic solvent OS1 (or of the mixture of organic solvents OS1). Step b) is preferably carried out at a temperature between 5° C. and the boiling point of the organic solvent OS1 (or of the mixture of organic solvents OS1), preferentially between 20° C. and the boiling point of the organic solvent OS1 (or of the mixture of organic solvents OS1).

The fluorination step may be performed at a pressure P preferably between 0 and 16 bar abs.

This step is preferably performed by dissolving the compound of formula (A) in the organic solvent OS1 or the mixture of organic solvents OS1 prior to the step of reaction with the fluorinating agent, preferably with anhydrous HF.

The molar ratio x between the fluorinating agent, preferably anhydrous HF, and the compound of formula (A) used is preferably between 1 and 10 and advantageously between 1 and 5.

The fluorination step may be carried out in a closed environment or in an open environment; preferably, step b) is carried out in an open environment with in particular release of HCl in gas form.

The fluorination reaction typically leads to the formation of HCl, the majority of which can be degassed from the reaction medium (just like the excess HF if the fluorinating agent is HF), for example by stripping with an inert gas (such as nitrogen, helium or argon).

The compound of formula (I) may optionally be subjected to a distillation step.

The step of contacting an anhydrous stream F1 with a compound of formula (I) may be carried out with a compound of formula (I) resulting directly from the fluorination step or resulting from an additional step of distillation of the composition obtained at the end of the fluorination step.

The compound of formula (A) may be prepared by any means known to those skilled in the art, for example as described in WO2015/158979, WO2009/123328, or else by reaction between a chlorosulfonyl isocyanate with chlorosulfonic acid (US2013/331609).

Compound (A) may also be commercially available.

The present invention also relates to a process for preparing a compound of formula (III):

F—SO₂—N⁻—SO₂—R₁Li⁺ (III)

wherein R₁ is as defined above, said process comprising the process for preparing a compound of formula (II) as defined above.

The present invention preferably relates to a process for preparing a compound of formula (III), comprising:

i) the process for preparing a compound of formula (II) as defined above; and

ii) a step of cation exchange by contacting the compound of formula (II) with a lithium salt, in particular chosen from the group consisting of lithium fluorides, lithium chlorides, lithium carbonates, lithium hydroxides, lithium sulfates, lithium chlorates, lithium perchlorates, lithium nitrites, lithium nitrates, and mixtures thereof.

The abovementioned process may include a step of intermediate purification of the compound of formula (II) prior to the step ii) of cation exchange. The purification may comprise a filtration step, a wash step with an organic solvent, an extraction step, etc.

Step ii) may be carried out in an organic solvent, which is preferably polar, or an aqueous solvent such as for example water.

Examples of polar organic solvents include alcohols, nitriles, carbonates, and mixtures thereof. For example, these may be methanol, ethanol, acetonitrile, dimethyl carbonate, ethyl methyl carbonate, and mixtures thereof.

The lithium salt may be a solid lithium salt or a lithium salt in solution in at least one organic solvent.

Reaction ii) may be carried out at a temperature between 0° C. and the boiling point of the solvent used, preferably between 0° C. and 50° C.

The reaction time for step ii) may be for example between 1 hour and 5 days, preferably between 1 hour and 1 day.

The molar ratio between the lithium salt and the compound of formula (II) may be between 0.9 and 5.

The abovementioned process may comprise a step iii) of recovering the product of formula (III).

Depending on the lithium salt used, the reaction medium can be filtered to remove the precipitate formed with the ammonium cation. The filtrate can then be concentrated to remove the solvent. A precipitate with the ammonium cation may form again and can be removed by filtration. The excess lithium salt can be removed by washing with water, which can be carried out after an evaporation or directly on the solution of the compound of formula (III) in an organic solvent chosen from the following families: esters, ethers, chlorinated solvents or aromatic solvents, such as for example dichloromethane, acetonitrile, ethyl acetate, butyl acetate, diethyl ether, tetrahydrofuran.

In a first embodiment, the solution of the compound of formula (III) obtained at the end of step ii) can be evaporated, for example by a thin-film evaporator or by an atomizer or by a rotary evaporator. The compound of formula (III) thus obtained can be dissolved in an amount of water that can vary between 4/1 and 1/1 of the total mass of the compound of formula (III) with solvent. The product dissolved in the aqueous solution can then be extracted using an organic solvent chosen from the following families: esters, ethers, chlorinated solvents or aromatic solvents, such as for example dichloromethane, ethyl acetate, butyl acetate, diethyl ether, tetrahydrofuran.

In a second embodiment, the solution of the compound of formula (III) obtained can be washed with water. There may be multiple washes, in particular from 2 to 10, with amounts increasing or decreasing in the course of the washes. The amounts by mass of water used in the wash(es) are between 1/10 and 2 times the mass of product solution to be washed. The washed organic phase can then be evaporated, in particular with a thin-film evaporator or an atomizer or a rotary evaporator.

The compound of formula (III) obtained by the abovementioned process may be subjected to at least one purification step. This may be a means of purification well known to those skilled in the art, such as for example liquid-liquid extractions, recrystallization, etc.

In one embodiment, the compound of formula (III) is chosen from the following compounds: LiN(FSO₂)₂, LiNSO₂CF₃SO₂F, LiNSO₂C₂F₅SO₂F, LiNSO₂CHF₂SO₂F, LiNSO₂CH₂FSO₂F, LiNSO₂C₂HF₄SO₂F, LiNSO₂C₂H₂F₃SO₂F, LiNSO₂C₂H₃F₂SO₂F, LiNSO₂C₃F₇SO₂F, LiNSO₂C₃H₂F₅SO₂F, LiNSO₂C₃H₄F₃SO₂F, LiNSO₂C₄F₉SO₂F, LiNSO₂C₄H₂F₇SO₂F, LiNSO₂C₄H₄F₅SO₂F, LiNSO₂C₅F₁₁SO₂F, the compound of formula (III) preferably being LiN(FSO₂)₂.

The inventors have advantageously found that the performance of an intermediate step of preparation of a compound of formula (II) advantageously makes it possible to prepare a compound of formula (III), such as LiFSI, at low cost and with high yield. More particularly, this process advantageously makes it possible to avoid a step of neutralization, with a lithium-based aqueous solution, of the compound of formula (I), for example bis(fluorosulfonyl)imide, which is unstable in aqueous solution, and therefore makes it possible to avoid generating degradation products likely to impact the performance of the final product.

In the context of the invention, the terms “between x and y” and “ranging from x to y” are understood as meaning a range inclusive of the limits x and y. For example, the temperature “between 30 and 100° C.” specifically includes the values 30° C. and 100° C.

All the embodiments described above may be combined with one another. In particular, each embodiment of any one step of the process of the invention may be combined with another particular embodiment.

1-15. (canceled)

16. A process for preparing a compound of the following formula (II):
F—SO₂—N⁻—SO₂—R₁NH₄⁺ (II),
wherein R₁ represents F or a linear or branched alkyl radical substituted by at least one fluorine atom, said process comprising a step of contacting an anhydrous stream F1 comprising ammonia (NH₃) with a compound of formula (I):
F—SO₂—NH—SO₂—R₁ (I)
R¹ being as defined above.

17. The process as claimed in claim 16, wherein R¹ represents one of the following radicals: F, CF₃, CHF₂, CH₂F, C₂HF₄, C₂H₂F₃, C₂H₃F₂, C₂F₅, C₃F₇, C₃H₂F₅, C₃H₄F₃, C₄F₉, C₄H₂F₇, C₄H₄F₅, or C₅F₁₁.

18. The process as claimed in claim 16, wherein the stream F1 is a gaseous stream comprising gaseous ammonia (NH₃).

19. The process as claimed in claim 16, wherein the stream F1 is a liquid stream comprising liquid ammonia (NH₃) or comprising a solution of ammonia (NH₃) in an organic solvent or mixture of organic solvents.

20. The process as claimed in claim 19, wherein the organic solvent is chosen from the group consisting of esters, nitriles, ethers, amines, phosphines, and mixtures thereof.

21. The process as claimed in claim 19, wherein the organic solvent is chosen from the group consisting of methyl acetate, ethyl acetate, butyl acetate, acetonitrile, propionitrile, isobutyronitrile, glutaronitrile, dioxane, tetrahydrofuran, methanol, ethanol, propanol, butanol, and mixtures thereof.

22. The process as claimed in claim 16, wherein the process comprises a preliminary step of dissolving gaseous or liquid NH₃ in an organic solvent or mixture of organic solvents.

23. The process as claimed in claim 19, wherein the concentration of ammonia (NH₃) dissolved in an organic solvent or a mixture of organic solvents is between 0.01 mol/L and the maximum solubility of ammonia in said organic solvent(s).

24. The process as claimed in claim 16, wherein the reaction step is performed at a temperature T ranging from 0° C. to 40° C.

25. The process as claimed in claim 16, wherein the molar ratio of the compound of formula (I) to ammonia is between 0.01 and 1.

26. The process as claimed in claim 16, wherein the compound of formula (I) is obtained by a process comprising a step of fluorination of a compound of formula (A):
Cl—(SO₂)—NH—(SO₂)—R² (A)
wherein R² represents one of the following radicals: Cl, F, CF₃, CHF₂, CH₂F, C₂HF₄, C₂H₂F₃, C₂H₃F₂, C₂F₅, C₃F₇, C₃H₄F₃, C₃HF₆, C₄F₉, C₄H₂F₇, C₄H₄F₅, C₅F₁₁, C₆F₁₃, C₇F₁₅, C₈F₁₇ or C₉F₁₉;
with at least one fluorinating agent.

27. The process as claimed in claim 26, wherein the fluorinating agent is chosen from the group consisting of HF, KF, AsF₃, BiF₃, ZnF₂, SnF₂, PbF₂, CuF₂, and mixtures thereof.

28. A process for preparing a compound of formula (III):
F—SO₂—N⁻—SO₂—R₁Li⁺ (III)
comprising a step of preparing a compound of formula (II) as claimed in claim 16.

29. The process as claimed in claim 28, wherein it comprises:
preparing the compound of formula (II)
F—SO₂—N⁻—SO₂—R₁NH₄⁺ (II),
wherein R₁ represents F or a linear or branched alkyl radical substituted by at least one fluorine atom; and
cation exchange by contacting the compound of formula (II) with a lithium salt chosen from the group consisting of lithium fluorides, lithium chlorides, lithium carbonates, lithium hydroxides, lithium sulfates, lithium chlorates, lithium perchlorates, lithium nitrites, lithium nitrates, and mixtures thereof.

30. The process as claimed in claim 28, wherein the compound of formula (III) is LiN(FSO₂)₂, LiNSO₂CF₃SO₂F, LiNSO₂C₂F₅SO₂F, LiNSO₂CHF₂SO₂F, LiNSO₂CH₂FSO₂F, LiNSO₂C₂HF₄SO₂F, LiNSO₂C₂H₂F₃SO₂F, LiNSO₂C₂H₃F₂SO₂F, LiNSO₂C₃F₇SO₂F, LiNSO₂C₃H₂F₅SO₂F, LiNSO₂C₃H₄F₃SO₂F, LiNSO₂C₄F₉SO₂F, LiNSO₂C₄H₂F₇SO₂F, LiNSO₂C₄H₄F₅SO₂F, LiNSO₂C₅F₁₁SO₂F.
